(19) United States
(12) Patent Application Publication
(10) Pub. No.: US 2025/0279640 A1
(43) Pub. Date: Sep. 4, 2025

(54) SUPERCONDUCTING WIRE STRUCTURE, SUPERCONDUCTING COIL, AND SUPERCONDUCTING DEVICE

(71) Applicant: KABUSHIKI KAISHA TOSHIBA, Tokyo (JP)

(72) Inventors: Keiko ALBESSARD, Yokohama Kanagawa (JP); Yasushi HATTORI, Kawasaki Kanagawa (JP); Tomoko EGUCHI, Shinagawa Tokyo (JP)

(73) Assignee: KABUSHIKI KAISHA TOSHIBA, Tokyo (JP)

(21) Appl. No.: 18/975,214

(22) Filed: Dec. 10, 2024

(30) Foreign Application Priority Data
Mar. 1, 2024 (JP) 2024-031264

(51) Int. Cl. H02G 15/08; A61N 5/10; B61B 13/08; G01R 33/3815; H01B 12/02; H01F 6/06

(52) U.S. Cl. CPC H02G 15/08; H01B 12/02; H01F 6/06; A61N 5/1077; A61N 2005/1087; B61B 13/08; G01R 33/3815

(57) ABSTRACT

A superconducting wire structure of embodiments includes: a first superconducting wire; a second superconducting wire adjacent to the first superconducting wire; a joint layer electrically connecting the first superconducting wire and the second superconducting wire; and a connecting member connecting the joint layer and at least one of the first superconducting wire and the second superconducting wire. The connecting member has a melting point equal to or more than 900° C. and equal to or less than 1100° C.

### CROSS-REFERENCE TO RELATED APPLICATION

This application is based upon and claims the benefit of priority from Japanese Patent Application No. 2024-031264, filed on Mar. 1, 2024, the entire contents of which are incorporated herein by reference.

### FIELD

Embodiments described herein relate generally to a superconducting wire structure, a superconducting coil, and a superconducting device.

### BACKGROUND

For example, in a nuclear magnetic resonance (NMR) apparatus or a magnetic resonance imaging (MRI) apparatus, a superconducting coil is used to generate a strong magnetic field. The superconducting coil is formed by winding a superconducting wire around a winding frame.

In order to lengthen the superconducting wire, for example, a plurality of superconducting wires are connected to each other. For example, the ends of two superconducting wires are connected to each other by using a connector. The connector for connecting the superconducting wires to each other is required to have a low electrical resistance and a high mechanical strength.

### BRIEF DESCRIPTION OF THE DRAWINGS

FIG. 1 is a schematic cross-sectional view of a superconducting wire structure according to a first embodiment;

FIG. 2 is a schematic cross-sectional view of an example of a method for measuring mechanical strength in a superconducting wire structure according to the first embodiment;

FIG. 3 is a schematic cross-sectional view of a first modification of a superconducting wire structure according to the first embodiment;

FIG. 4 is a schematic cross-sectional view of a second modification of a superconducting wire structure according to the first embodiment;

FIG. 5 is a schematic cross-sectional view of a third modification of a superconducting wire structure according to the first embodiment;

FIG. 6 is a schematic cross-sectional view of a fourth modification of a superconducting wire structure according to the first embodiment;

FIG. 7 is a schematic cross-sectional view of another example of a method for measuring mechanical strength in a superconducting wire structure according to the first embodiment;

FIG. 8 is a schematic cross-sectional view of a fifth modification of a superconducting wire structure according to the first embodiment;

FIG. 9 is a schematic cross-sectional view of a sixth modification of a superconducting wire structure according to the first embodiment;

FIG. 10 is a schematic cross-sectional view of further example of a method for measuring mechanical strength in a superconducting wire structure according to the first embodiment;

FIG. 11 is a schematic cross-sectional view of a seventh modification of a superconducting wire structure according to the first embodiment;

FIG. 12 is a schematic perspective view of a superconducting coil according to a second embodiment;

FIG. 13 is a schematic cross-sectional view of the superconducting coil according to the second embodiment; and

FIG. 14 is a block diagram of a superconducting device according to a third embodiment.

### DETAILED DESCRIPTION

Hereinafter, embodiments will be described with reference to the drawings. In the following description, constituents exhibiting the same or similar function are denoted by the same reference signs throughout all drawings, and an overlapping description will be omitted.

Each drawing is a schematic view to facilitate the description and understanding of the embodiments, and the shape, size, ratio, and the like thereof may be different from those of an actual device, but these embodiments can be appropriately designed and modified by taking into consideration the following description and known techniques.

In this specification, the “particle diameter” of each particle or the like is the long diameter of the particle unless otherwise specified. The long diameter of the particle is the maximum length among the lengths between any arbitrary two points on the circumference of the particle. In addition, the short diameter of the particle is the length of a line segment that passes through the midpoint of the long diameter, is perpendicular to the long diameter, and has both ends at the circumference of the particle.

In addition, the aspect ratio of the particle is the ratio of the long diameter to the short diameter (long diameter/short diameter) of the particle. The long and short diameters of the particle can be calculated, for example, by image analysis of scanning electron microscope (SEM) images.

### First Embodiment

A superconducting wire structure according to a first embodiment includes: a first superconducting wire; a second superconducting wire adjacent to the first superconducting wire; a joint layer electrically connecting the first superconducting wire and the second superconducting wire; and a connecting member connecting the joint layer and at least one of the first superconducting wire and the second superconducting wire. The connecting member has a melting point equal to or more than 900° C. and equal to or less than 1100° C.

FIG. 1 is a schematic cross-sectional view of a superconducting wire structure 200 according to the first embodiment. A superconducting wire structure 200 according to the first embodiment, for example, is used to lengthen superconducting wires by joining two superconducting wires (a first wire 210 and a second wire 220) to each other.

Here, a first direction D1 crossing a direction from the first wire 210 to the joint layer 221 and along the first wire 210 is defined as an X-axis direction. A second direction D2 from the first wire 210 to the joint layer 221 is defined as a Z-axis direction. One direction perpendicular to the Z-axis direction is defined as an X-axis direction. A direction perpendicular to the Z-axis direction and the X-axis direction is defined as a Y-axis direction.

The superconducting wire structure 200 includes the first wire 210, the second wire 220, the joint layer 221, and a connecting member 222.

The connecting member 222 connects the joint layer 221 and at least one of the first wire 210 and the second wire 220.

In FIG. 1, the connecting member 222 is in contact with a part of a lower surface of the joint layer 221 having an interface with a part of the first wire 210 and a part of the second wire 220. Thus, the joint layer 221, the first wire 210, and the second wire 220 are physically connected through the connecting member 222. The connecting member 222 is preferably provided to connect the joint layer 221, the first wire 210, and the second wire 220 because it is possible for mechanical strength of the superconducting wire structure 200 to be improved.

The connecting member 222 is, for example, metal. The connecting member 222 includes at least one element selected from the group composing of Ag, Cu, Au, and Ge.

The connecting member 222 has a melting point equal to or more than 900° C. and equal to or less than 1100° C. In manufacturing the superconducting wire structure 200 according to the embodiment, a heat treatment with the superconducting wire structure 200 pressurized is, for example, equal to or more than 700° C. and equal to or less than 850° C. Thus, the temperature of the heat treatment is equal to or more than 70% of the melting point of the connecting member 222. Hence, it is possible for the connecting member 222 to be easily softened during the heat treatment.

Since the melting point of the connecting member 222 is equal to or more than 900° C., it becomes possible to prevent particles composing the joint layer 221 from being extruded when the connecting member 222 melts during a heat treatment of the superconducting member 200 with the superconducting wire structure 200 being pressurized. Therefore, it becomes possible to prevent formation of a region where particles do not exist in the joint layer 221. Thus, it becomes possible for a sufficient path for current in the joint layer 221 to be obtained. Furthermore, in the joint layer 221 according to the embodiment, the heat treatment is performed while the particles are in contact with each other, causing a reaction between the particles which makes it possible to obtain a sufficient path for current in the joint layer 221. Accordingly, raising the temperature of the heat treatment over the melting point of the connecting member 222 makes it possible to prevent a portion of the melting connecting member 222 from entering between the particles in the joint layer 221 before the particles react in the joint layer 221.

In addition, since the melting point of the connecting member 222 is equal to or less than 1100° C., it becomes possible for the temperature of the heat treatment to be equal to or more than 70% of the melting point of the connecting member 222. Thus, it is possible for the connecting member 222 to contribute to the connection of each wire 210 and 220.

Hereinafter, another aspect of the superconducting wire structure 220 according to an embodiment will be described.

### (First Wire)

The first wire 210 includes a first conducting layer 210a, and a first substrate 210b. The first conducting layer 210a is provided on the first substrate 210b.

The first wire 210 is electrically connected to the second wire 220 through the joint layer 221. Therefore, it is possible for the current to flow between the first wire 210 and the second wire 220 through the joint layer 221.

The first substrate 210b is, for example, a metal. The first substrate 210b is, for example, a nickel alloy or a copper alloy.

The first substrate 210b is, for a specific example, a nickel-chromium-molybdenum alloy.

The first superconducting layer 210a is, for example, an oxide superconducting layer. The first superconducting layer 210a contains, for example, a rare earth element (RE), barium (Ba), copper (Cu), and oxygen (O). For example, the first superconducting layer 210a contains at least one rare earth element (RE) in a group composed of: yttrium (Y), lanthanum (La), neodymium (Nd), samarium (Sm), europium (Eu), gadolinium (Gd), terbium (Tb), dysprosium (Dy), holmium (Ho), erbium (Er), thulium (Tm), ytterbium (Yb), and lutetium (Lu).

The first superconducting layer 210a has, for example, a chemical composition represented by (RE) Ba₂Cu₃O_{δ} (RE is a rare earth element, 6≤δ≤7). Specifically, the first superconducting layer 210a has a chemical composition represented by, for example, GdBa₂Cu₃O_{δ} (6≤δ≤7), YBa₂Cu₃O_{δ} (6≤δ≤7), or EuBa₂Cu₃O_{δ} (6≤δ≤7). The first superconducting layer 210a contains, for example, a rare earth oxide superconductor.

The first superconducting layer 210a contains, for example, a single crystal having a perovskite structure.

### (Second Wire)

The second wire 220 is provided adjacent to the first superconducting wire 210.

The second wire 220 includes a second superconducting layer 220a, and a second substrate 220b. The second superconducting layer 220a is provided on the second substrate 220b.

The second substrate 220b is, for example, a metal as the first substrate 210b. The second substrate 220b may include the same material as the first substrate 210b or different materials.

The second superconducting layer 220a is, for example, an oxide superconducting layer similar as the first superconducting layer 210a. The second superconducting layer 220a may include the same material as the first substrate 210a or different materials.

### (Joint Layer)

The joint layer 221 is in a contact with at least a part of the first wire 210 and at least a part of the second wire 220. The joint layer 221 electrically connects the first wire 210 and the second wire 220.

The joint layer 221 is connected to one of the first wire 210 and the second wire 220 through the connecting member 222. For example, the connecting member 222 connects a side surface of the first wire 210 crossing a first direction from the first wire 210 to the second wire 220 with a lower surface of the joint layer 221 having an interface with the first wire 210. Here, for example, the first direction is the X-axis direction. Since the connecting member 222 connects the exposed side surface of the first wire 210 with the exposed lower surface of the joint layer 221, it is possible to prevent peeling of the particles composing the joint layer 221 from the first wire 210 caused by entry of the connecting member 222 to the joint layer 221. Therefore, it is possible to prevent deterioration of the conduction path.

The joint layer 221 contains, for example, single crystal particle groups containing a rare earth element (RE), barium (Ba), copper (Cu), and oxygen (O) or polycrystal particle groups containing the same elements as the single crystal. The joint layer 221 is, for example, composed of a particle group of a single crystal containing at least one rare earth element (RE) in a group composing of yttrium (Y), lanthanum (La), neodymium (Nd), samarium (Sm), europium (Eu), gadolinium (Gd), terbium (Tb), dysprosium (Dy), holmium (Ho), erbium (Er), thulium (Tm), ytterbium (Yb), and lutetium (Lu) or polycrystal containing the same elements as the single crystal.

The joint layer 221 is, for example, composed of a particle group of a single crystal having a chemical composition represented by (RE) Ba₂Cu₃O_{δ} (RE is a rare earth element, 6≤δ≤7) or polycrystal having the same chemical composition as the single crystal. Specifically, the joint layer 221 is composed of a particle group of a single crystal having a chemical composition represented by, for example, GdBa₂Cu₃O_{δ} (6≤δ≤7), YBa₂Cu₃O_{δ} (6≤δ≤7), or EuBa₂Cu₃O_{δ} (6≤δ≤7) or polycrystal having the same chemical composition as the single crystal. The joint layer 221 is, for example, composed of a particle group of a single crystal of a rare earth oxide superconductor or polycrystal of the rare earth oxide superconductor.

The average particle size of particles in these particle groups is equal to or more than 500 nm and equal to or less than 30 μm, preferably equal to or more than 1 μm and equal to or less than 10 μm. When the average particle size of particles in these particle groups is equal to and more than 500 nm, sufficient crystallinity can be obtained, and thus, high superconducting properties can be obtained. Further, when the particle size is equal to and less than 30 μm, the thickness of the joint layer 221 can be controlled so as not to be increased. This can reduce portions that become resistance portions such as voids existing in joint layer 221, and thus, the resistance of the superconducting wire structure 200 can be reduced. In addition, when the particle size is equal to or less than 30 μm, the contact area between the joint layer 221 and the first wire 210 and the contact area between the joint layer 221 and the second wire 220 can be sufficiently increased, and thus, the mechanical strength of the superconducting wire structure 200 can be increased.

The aspect ratio of these particle groups is equal to or more than 1.5 and equal to or less than 50, preferably equal to or more than 3 and equal to or less than 10. When the aspect ratio is equal to or more than 1.5, the joint layer 221 can have a characteristic as superconductivity. In addition, when the aspect ratio is equal to or less than 50, the bulk density of the joint layer 221 can be sufficiently increased, and the thickness of the joint layer 221 can be controlled so as not to be increased. Furthermore, the contact area between the joint layer 221 and the first wire 210 and the contact area between the joint layer 221 and the second wire 220 can be sufficiently increased.

The porosity of the joint layer 221 is equal to or more than 10% and equal to or less than 70%, preferably equal to or more than 20% and equal to or less than 50%. When the porosity is equal to or more than 10%, a part of the connecting member 222 easily enters the region that exists in the vicinity of the interfacial surface between the first superconducting wire 210 and the joint layer 221, so that the mechanical strength can be increased. When the porosity is equal to or less than 70%, the amount of particles in the joint layer 221 can be made sufficient, so that a decrease in critical current due to a shortage of current paths can be prevented.

Here, a method for manufacturing the superconducting wire structure according to the embodiment in FIG. 1 will be described.

First, the first wire 210 and the second wire 220 are prepared. Here, when each superconducting layer has a protection layer (mainly a Cu layer and an Ag layer) for preventing the superconducting layer from reacting with moisture in the atmosphere, for example, this layer is removed by using a wet etching method, and each of the superconducting layer 210a and 220a is exposed.

Thereafter, the superconducting layers of the first wire 210 and the second wire 220 are configured to be oriented in the same direction. In addition, the connecting member 222 is arranged so that at least a part of each wire is in contact with the connecting member 222. Here, the connecting member 222 is obtained by using, for example, a metal sheet. The connecting member 222 to be used is preferably equal to or more than 0.001 mm and equal to or less than 1 mm in thickness. When the thickness of the connecting member 222 is equal to or more than 0.001 mm, sufficient strength of superconducting wire structure 200 can be obtained. In addition, since the thickness is equal to or less than the 1 mm, heat generated during the heat treatment sufficiently reaches the inside of the connecting member 222, and the connecting member 222 can be easily softened. Therefore, the first wire 210 and the second wire 220 can be connected to each other by the connecting member 222.

Then, the single crystal or polycrystal particles of the rare earth oxide superconductor described in the section above of (Joint Layer) and a solution containing an organic compound containing the same kind of metal element as a main component are mixed at a ratio of 5:1 to 1:3 depending on the average particle diameter of the particles, to obtain a mixture for the joint layer 221. The mixture obtained as described above is molded on the first wire 210 and the second wire 220 to have a necessary thickness. Then, drying is performed, and baking is performed in oxygen at equal to or more than 700° C. and equal to or less than 850° C., so that the joint layer 221 is formed on the first wire 210 and the second wire 220.

In this way, a structure for obtaining the superconducting wire structure 200 in FIG. 1 is obtained.

Next, in the structure obtained as described above, a weight is placed on a portion where the joint layer 221 and each wire 210 and 220 are overlapped with each other to apply pressure. For example, a jig for pressurization is prepared and the structure is sandwiched between the jig to apply pressure. The jig may be removed after the connection, or the jig may be left attached. The jig is preferably removed because the superconducting wire structure 200 can be easily wound around a coil.

Next, heat treatment is performed in a pressurized state. The heat treatment is performed, for example, in argon containing O₂ having concentration of equal to or more than 100 ppm and equal to or less than 1500 ppm at a temperature of equal to or more than 700° C. and equal to or less than 850° C., and then, in O₂ at a temperature of equal to or more than 300° C. equal to or less than to 600° C.

The connecting member 222 is softened by the heat treatment, and the connecting member 222 as shown in FIG. 1 is obtained.

In this way, superconducting wire structure 200 in FIG. 1 is obtained.

The critical current of the superconducting wire structure 200 according to the present embodiment can be measured, for example, as follows.

### <Measurement of Critical Current>

The critical current is the maximum current value that can be passed at zero electrical resistance, and it can be measured by a four terminal measurement method. The terminal is attached to the superconducting wire structure 200 after the connection, and measurement is performed in liquid nitrogen. Since the voltage increases as the resistance increases, the current value at which the voltage starts to increase is defined as the critical current.

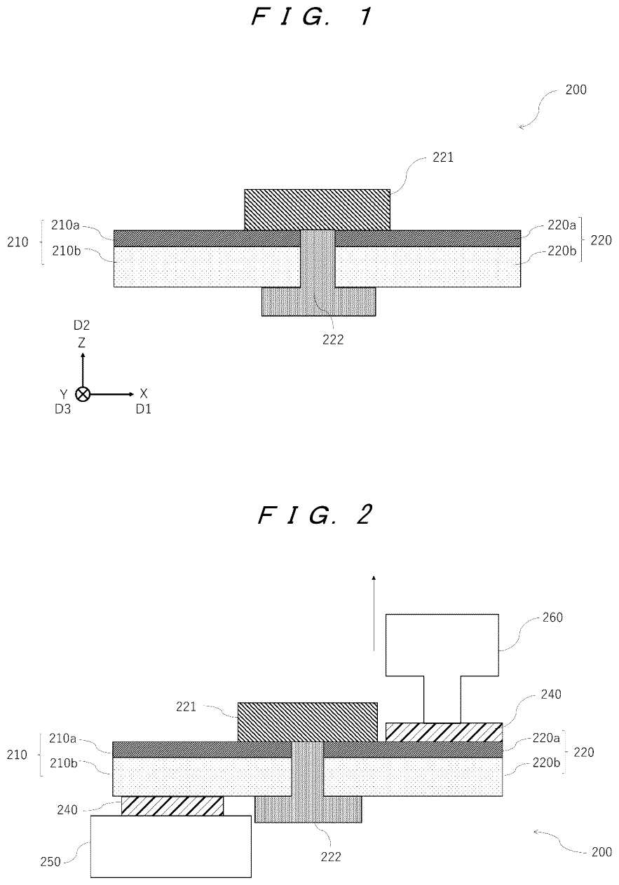

The mechanical strength of the superconducting wire structure according to the present embodiment can be evaluated, for example, according to Japanese Industrial Standards JIS K 6849 (1994) “Testing methods for tensile strength of adhesive bonds (method of measuring by tensile load perpendicular to the bonding surface)”. Each specific measurement method in the example of FIG. 1 will be described with reference to FIG. 2. FIG. 2 is a schematic cross-sectional view of an example of a method for measuring mechanical strength in the example of the superconducting wire structure shown in FIG. 1.

### <Method for Measuring Mechanical Strength of Superconducting Wire Structure in FIG. 1>

### (Fixing of Superconducting Wire Structure)

In FIG. 2, one surface of a double-sided adhesive tape 240 is bonded to the first substrate 210b or the second substrate 220b (in the example shown to the substrate 210b). In addition, the other surface of the double-sided adhesive tape 240 is adhered to a pedestal 250 and the superconducting wire structure 200 structure is fixed to the pedestal 250. At this time, the double-sided adhesive tape 240 is prepared so that the width of the tape 240 is the same length as the wire for fixing the pedestal 250 and the substrate of one of the wires. Further, the length of the tape 240 is a length that enables strong enough adhesion that does not cause peeling between the pedestal 250 and the substrate during measurement of the mechanical strength.

Next, one surface of another double-sided adhesive tape 240 is bonded to at least a part of the superconducting layer of the wire that is not fixed to the pedestal 250. In the case of FIG. 2, the superconducting layer of the wire is the second superconducting layer 220a of the second wire 220. At this time, the double-sided adhesive tape 240 is prepared so that the width of the tape 240 is the same length as the wire for fixing the superconducting layer of the other wire and the tensile device 260. Further, the length of the tape 240 is a length that enables strong enough adhesion that does not cause peeling between the pedestal 250 and the substrate during measurement of the mechanical strength. In addition, the double-sided adhesive tape 240 used for fixing the tensile device 260 and the wire is provided at a certain distance from the end of the joint layer 221. For example, the distance can be equal to or more than 1 mm and equal to or less than 40 mm, preferably equal to or more than 5 mm and equal to or less than 20 mm.

The double-sided adhesive tape 240 may be any tape as long as it has a sufficient adhesive strength to withstand the measurement, and for example, a carbon tape for SEM observation can be used.

When the mechanical strength of the superconducting wire structure for the example of FIG. 2 is measured, the double-sided adhesive tape 240 having the above-described size is used for each measurement.

### (Measurement Using Tensile Device)

As the operation of the tensile device 260, the tensile device 260 is operated in a direction away (the direction of the arrow in FIG. 2) from the wire (the second wire 220 in FIG. 2) fixed to the tensile device 260 via the double-sided adhesive tape 240. At this time, the operation speed of the tensile device 260 is set to be constant, for example, equal to or more than 100 μm/s and equal to or less than 5 mm/s, preferably equal to or more than 500 μm/s and equal to or less than 1 mm/s. Since the operation of the tensile device 260 is equal to or more than 100 μm/s and equal to or less than 5 mm/s, the measurement time can be shortened within a range in which measurement errors due to the wires being immediately peeled off are suppressed. As the measurement, for example, the distance by which the tensile device 260 moves until the wire is completely peeled off from the joint layer 221 can be measured as the mechanical strength.

The mechanical strength between the first wire 210 or the second wire 220 and the connection layer 221 can be evaluated by the above-described method.

The porosity of joint layer 221 of superconducting wire structure 200 can be measured as follows after the above-described mechanical strength test.

### <Method for Measuring Porosity of Joint Layer>

First, the connection portion including the joint layer 221 is embedded in, for example, an epoxy-based resin, and the vicinity of the center of the connection portion which is firmly connected is cut by a diamond saw or the like because peeling is a concern at the end of the connection. At this time, in a modification example described later where the connecting member 222 is, for example, disposed between the joint layer 221 and the first wire 210, the cross section for measuring the porosity of the joint layer 221 is located at a position avoiding the connecting member 222. In addition, in a modification example described later where the connecting member 222 has entered the joint layer 221, the porosity of the joint layer 221 is measured after the connecting member 222 is measured.

Next, the cut surface is polished with waterproof paper and buffed or the like, and then coated with a conductive material, and the observation positions are determined by SEM. Ion milling is performed on the determined observation positions, and SEM observation is performed after conductive coating is performed.

Although depending on the average particle diameter of the powder used, when the average particle diameter is equal to or more than 2 μm and equal to or less than to 3 μm, SEM images of three positions in the cross section are taken at a magnification of about 2500 times. At this time, the joint layer 221 is processed to have three cross sections at positions where the sides corresponding to the long sides of the substantially quadrangular shape of the joint layer 221 in plan view are equally divided into four. The magnification of the SEM image is set such that the larger the average particle diameter, the lower the magnification, and the smaller the average particle diameter, the higher the magnification. The SEM image is binarized to separate particles from voids, for example, using ImageJ, and then the areas of the particles and the voids are calculated. The porosity in each cross section is calculated from the ratio of the areas. The porosity obtained in each cross section is averaged to obtain the porosity of the joint layer 221 to be obtained.

Hereinafter, a modification of the superconducting wire structure according to the first embodiment will be described.

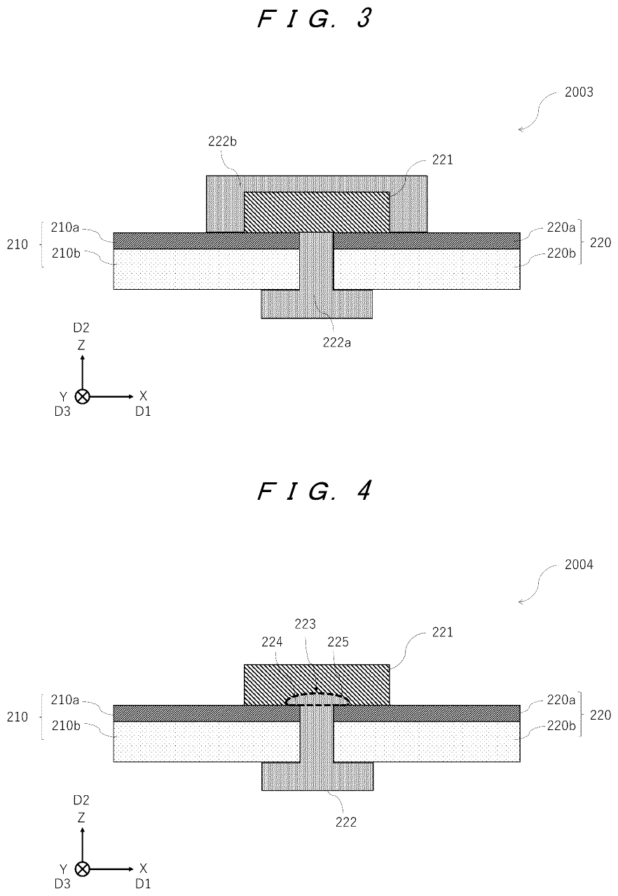

FIG. 3 is a schematic cross-sectional view of a first modification of the superconducting wire structure according to the first embodiment.

In FIG. 3, the connecting member includes a connecting member 222a positioned between the first wire 210 and the second wire 220. In FIG. 3, the connecting member further includes a connecting member 222b covering a part, except for the interfaces between the first wire 210 and the second wire 220, exposed to the outside of the joint layer 221.

The connecting member 222b is in contact with at least a part of the first superconducting layer 210a and at least a part of the second superconducting layer 220a. This improves the mechanical strength between the joint layer 221 and the first wire 210 and the mechanical strength between the joint layer 221 and the second wire 220, and thus the mechanical strength of the superconducting wire structure 2003 can be improved.

When a surface of the joint layer 221 facing the lower surface having the interface with each wire 210 and 220 is defined as an upper surface and a surface of the joint layer 221 located between the upper surface and the lower surface of the joint layer 221 is defined as a side surface, the connecting member 222b is not necessarily in contact with the entire upper surface and the entire side surfaces.

The connecting member 222b may be provided such that the joint layer 221 is in contact with either or both the first wire 210 and the second wire 220. For example, there may be a region where a part of the side surface of the joint layer 221 is not covered. In addition, a microscopic gap may be present between the connecting member 222b and the joint layer 221. The connecting member 222b is preferably in contact with substantially the entire upper surface and the entire side surfaces of the joint layer 221.

The superconducting wire structure 2003 in FIG. 3 can be manufactured as follows. Here, the description of the same parts as those of the method of manufacturing the superconducting wire structure 200 in FIG. 1 will be omitted.

First, after the above-described method for manufacturing a structure for obtaining the superconducting wire structure 200 of FIG. 1 is performed, the connecting member 222b is disposed to be in contact with the joint layer 221. Thereafter, the heat treatment with the above-described structure being pressurized is performed to obtain the superconducting wire structure 2003 in FIG. 3.

The critical current, the mechanical strength, and the porosity of the joint layer 221 of the obtained superconducting wire structure 2003 of FIG. 3 can be measured in the same manner as the methods described above in <Measurement of Critical Current>, <Method for Measuring Mechanical Strength of Superconducting Wire Structure in FIG. 1>, and <Method for Measuring Porosity of Joint Layer>.

FIG. 4 is a schematic cross-sectional view of a second modification of the superconducting wire structure according to the first embodiment.

In this further embodiment the connecting member 222 is also present inside the joint layer 221 in FIG. 4. The region 223 is a portion where a part of the connecting member 222 exists inside the joint layer 221. The region 223 is generated by, for example, a part of the connecting member 222 entering the joint layer 221. This is because the joint layer 221 is formed of the particle groups, and the region 223 is generated by a part of the connecting member 222 entering a void area or areas present between the particle groups. This can improve the mechanical strength of superconducting wire structure 2004, and thus the region 223 is preferably present.

The connecting member 222 covers a part of the surface of the first substrate 210b and a part of the surface of the second substrate 220b

The superconducting wire structure 2004 in FIG. 4 can be manufactured in the same manner as the method for fabricating superconducting wire structure 200 in FIG. 1 described above.

In FIG. 4, the connecting member 222 is softened by the heat treatment, whereby the connecting member 222 enters the joint layer 221 and the region 223 is formed.

The region 223 includes at least one of a first region 224 present between the joint layer 221 and the first wire 210, and a second region 225 present between the joint layer 221 and the second wire 220. In this case, the first region 224 and the second region 225 satisfy at least one of a first condition and a second condition. In the first condition, the first region 224 is located in a range of equal to or more than 5 μm and equal to or less than 1 mm from an end of the first wire 210 in the first direction D1. The second condition is that the second region 225 is present in a range of equal to or more than 5 μm and equal to or less than 1 mm from an end of the second wire 220 in the first direction D1. Preferably, the first condition and the second condition are satisfied. That is, it is preferable that the first region 224 is present in a range of equal to or more than 5 μm and equal to or less than 1 mm from the end of the first wire 210, and the second region 225 is present in a range of equal to or more than 5 μm and equal to or less than 1 mm from the end of the second wire 220. These can improve the mechanical strength of the superconducting wire structure 2004.

Preferably, at least one of the first condition and the second condition is satisfied, and the connecting member 222 existing in the first region 224 and the first wire 210 are in contact with each other, or the connecting member 222 existing in the second region 225 and the second wire 220 are in contact with each other. Thereby, the mechanical strength of the superconducting wire structure 2004 can be improved.

The region 223 is preferably present in the range of equal to or less than 1 mm from the interface between the first wire 210 or the second wire 220 and the joint layer 221 also in the second direction D2. This can improve the mechanical strength of the superconducting wire structure 2004.

The upper limit of the range from the end of the first wire 210 or the end of the second wire 220 where the first region 224 or the second region 225 may be present is preferably equal to or less than 200 μm. Since the first region 224 or the second region 225 is present at a position equal to or more than 5 μm from the end of the first wire 210 or the end of the second wire 220, the mechanical strength as the superconducting wire structure 2004 can be increased. In addition, when the first region 224 or the second region 225 is present in the range of equal to or less than 1 mm from the end of the first wire 210 or the end of the second wire 220, the intrusion of the connecting member 222 in the joint layer 221 by pushing the particles composing of the joint layer 221 in the first direction can be suppressed. This makes it possible to obtain a sufficient connection area and to provide a sufficient current path, thereby it is possible to prevent a decrease in critical current. Here, the connection area is a contact area between the first wire 210 or the second wire 220 and the joint layer 221 when a current passes between the wires via the joint layer 221.

Therefore, to further increase the mechanical strength of superconducting wire structure 2004 and to achieve a higher critical current by obtaining sufficient connection areas, the range from the end of first wire 210 or the end of second wire 220 where first region 224 or second region 225 can exist is preferably equal to or more than 5 μm and equal to or less than 1 mm.

The critical current, the mechanical strength, and the porosity of the joint layer 221 of the obtained superconducting wire structure 2004 of FIG. 4 can be measured in the same manner as the methods described above in <Measurement of Critical Current>, <Method for Measuring Mechanical Strength of Superconducting Wire Structure in FIG. 1>, and <Method for Measuring Porosity of Joint Layer>. However, in the case where the length of the connecting member 222 along the first direction in which the connecting member 222 enters the joint layer 221 is measured, the measurement is performed before the measurement of the porosity of the joint layer 221.

Here, a method for measuring the length of the connecting member 222 entering the joint layer 221 along the first direction will be described.

### <Method for Measuring Length Along First Direction of Member Entering Joint Layer>

When the connecting member 222 includes the first region 224 as shown in FIG. 4, the distance can be measured by measuring the connecting member 222 remaining on the first superconducting layer 210a of the first wire 210 after the separation of the joint layer 221 and, for example, the first wire 210.

First, after <Method for Measuring Mechanical Strength of Superconducting Wire Structure in FIG. 1> is performed, the joint layer 221 and, for example, the first wire 210 are separated from each other.

Thereafter, a mapping image of elements derived from the components of the connecting member 222 is captured by EDX (Energy Dispersive X-ray Spectroscope) on the surfaces of the first superconducting layer 210a of the first wire 210. This makes it possible to measure the depth of the connecting member 222 entering the joint layer 221 in the first direction D1.

When capturing EDX images, first, the area on the surface of the first superconducting layer 210a, where the joint layer 221 existed before separation from the first wire 210, is divided into five equal parts along the third direction D3, which intersects with the first direction D1 and is parallel to the surface. The third direction D3 is, for example, the Y-axis direction along the surface of the first superconducting layer 210a, intersecting with the X-axis. The regions at both ends of the five equal parts along the Y-axis direction are excluded from the EDX imaging area. That is, the central three regions of the five equal parts along the third direction D3 are set as the EDX imaging area. The EDX imaging magnification is set to a range where the connecting member 222 with the longest distance along the first direction D1 is within the field of view in each imaging area. In each EDX image obtained in this way, the average length of the connecting member 222 in the third direction on the surface of the first superconducting layer 210a is determined. The average length of the invasion mentioned above is determined, for example, at positions dividing each width of the EDX images along the third direction D3 into six equal parts. By averaging these five lengths obtained from each of the three EDX images, a total of 15 lengths, the length of the connecting member 222 invading the joint layer 221 along the first direction D1 can be determined.

FIG. 5 is a schematic cross-sectional view of a third modification of the superconducting wire structure according to the first embodiment.

In FIG. 5, the connecting member 222 has a second connecting member 222b covering a portion exposed outside the joint layer 221, excluding the interface with the first wire 210 and the second wire 220 in addition to the first connecting member 222a located between the first wire 210 and the second wire 220.

The second connecting member 222b also may be present inside the joint layer 221, and the region 223 may exist in FIG. 5.

The superconducting wire structure 2005 in FIG. 5 can be manufactured in the same manner as the method of fabricating the superconducting wire structure 2003 in FIG. 3 described above.

The critical current, the mechanical strength, and the porosity of the joint layer 221 of the obtained superconducting wire structure 2005 of FIG. 5 can be measured in the same manner as the methods described in <Measurement of Critical Current>, <Method for Measuring Mechanical Strength of Superconducting Wire Structure in FIG. 1>, and <Method for Measuring Porosity of Joint Layer>.

In FIG. 5, the second connecting member 222b may include the region 223 by the second member 222b entering the joint layer 221 along the second direction D2. The region is preferably present in a range of equal to or more than 5 μm and equal to or less than 1 mm from the surface of the joint layer 221 in the second direction D2. The region can be identified by observing a cross section of the joint layer 221 in a plane intersecting with the X-Y plane formed by the X axis and the Y axis, for example, in the Z-X plane.

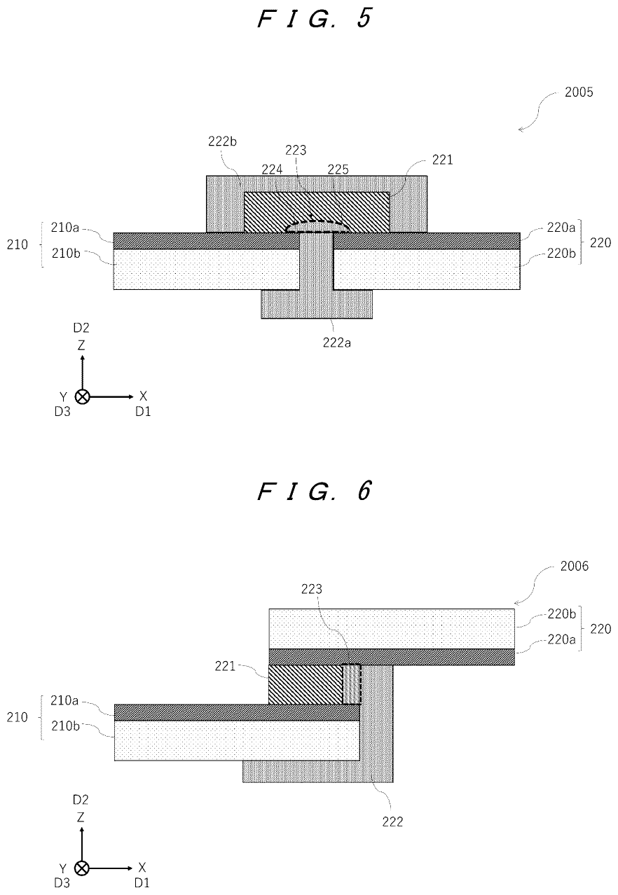

FIG. 6 is a schematic cross-sectional view of a fourth modification of the superconducting wire structure according to the first embodiment.

In FIG. 6, the first superconducting layer 210a and the second superconducting layer 220a are provided to face each other, and the joint layer 221 is provided to be positioned between the first superconducting layer 210a and the second superconducting layer 220a.

In this case, the superconducting layers of the first wire 210 and the second wire 220 face each other and the joint layer 221 is interposed therebetween, and thus the joint layer 221 is protected. For example, when the superconducting wire structure 2006 of FIG. 6 is wound into a coil, the joint layer 221 of the superconducting wire structure 2006 wound in the n-th turn is protected from contact with the superconducting wire structure 2006 in the (n+1)-th turn, and thus wear of the joint layer 221 can be suppressed.

The connecting member 222 connects the joint layer 221 to at least one of the first wire 210 and the second wire 220. In FIG. 6, the connecting member 222 is in contact with one surface of the joint layer 221.

The connecting member 222 enters the joint layer 221 and forms a region 223 that exists in a range of equal to or more than 5 μm and equal to or less than 1 mm from the end of the first wire 210.

The superconducting wire structure 2006 in FIG. 6 can be manufactured as follows.

Firstly, at least a part of the first substrate 210b and the connecting member 222 are disposed to be in contact with each other. Thereafter, the joint layer 221 is applied on at least a part of the second superconducting layer 220a. Secondly, at least a part of the first superconducting layer 210a, and in the joint layer 221 at least a part of a surface on the opposite side to the surface with which the second superconducting layer 220a is in contact, are provided to face each other. That is, the joint layer 221 is located between the first superconducting layer 210a and the second superconducting layer 220a.

Thus, a structure for obtaining the superconducting wire structure 2006 in FIG. 6 is obtained. Thereafter, the superconducting wire structure 2006 in FIG. 6 is obtained by the above-described pressurizing method and heat treatment.

The critical current, the mechanical strength, and the porosity of the joint layer 221 of the obtained superconducting wire structure 2006 of FIG. 6 can be measured in the same manner as the methods described above in <Measurement of Critical Current>, <Method for Measuring Mechanical Strength of Superconducting Wire Structure in FIG. 1>, and <Method for Measuring Porosity of Joint Layer>.

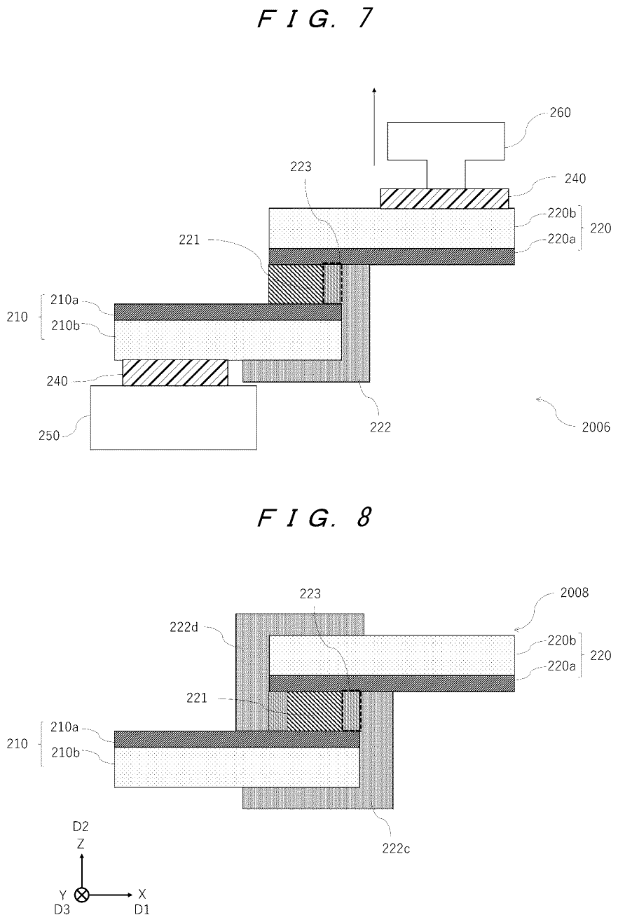

A method for measuring the mechanical strength in FIG. 6 will be described with reference to FIG. 7. Here, FIG. 7 is a schematic cross-sectional view showing a method of measuring the mechanical strength in the example of the superconducting wire structure 2006 shown in FIG. 6.

### <Method for Measuring Mechanical Strength of Superconducting Wire in FIG. 6>

In FIG. 7, the substrate of one wire and the pedestal 250 are fixed to each other via the double-sided adhesive tape 240 in the same manner as <Method for Measuring Mechanical Strength of Superconducting Wire Structure in FIG. 1>. Then, the substrate of the other wire and the tensile device 260 are fixed to each other via the double-sided adhesive tape 240. The dimensions of the double-sided adhesive tape 240 used in this case are the same as the dimensions described in <Method for Measuring Mechanical Strength of Superconducting Wire Structure in FIG. 1>. In the case of FIG. 7, the double-sided adhesive tape 240 is provided at a position at a certain distance from the end of the connecting member 222, and the distance is the same as the distance described in <Method for Measuring Mechanical Strength of Superconducting Wire Structure in FIG. 1>.

FIG. 8 is a schematic cross-sectional view of a fifth modification of the superconducting wire structure according to the first embodiment. In FIG. 8, the connecting member 222 includes a third connecting member 222c and a fourth connecting member 222d.

The third connecting member 222c and the fourth connecting member 222d connect the joint layer 221 and at least one of the first wire 210 and the second wire 220.

The fourth connecting member 222d is in contact with the other side surface of the connection layer 221 different from the side surface in contact with the third connecting member 222c in FIG. 8. The fourth connecting member 222d covers the other side surface of the connection layer 221, and thus, it is possible to further suppress the wear of the connection layer 221 due to the contact with the other superconducting wire structure 2008, compared to FIG. 6.

The superconducting wire structure 2008 in FIG. 8 can be manufactured as follows.

First, the first substrate 210b is disposed to be in contact with at least a portion of the third connecting member 222c. Thereafter, the joint layer 221 is applied on at least a part of the second superconducting layer 220a. Secondly, at least a part of the first superconducting layer 210a, and in the joint layer 221 at least a part of a surface on the opposite side to the surface with which the second superconducting layer 220a is in contact, are provided to face with each other. Thereafter, the second substrate 220b is disposed to be in contact with at least a portion of the fourth connecting member 222d. In this case, the connecting member 222c in contact with the first substrate 210b and the connecting member 222d in contact with the second substrate 220b can be formed using different materials or the same material.

Thus, a structure for obtaining the superconducting wire structure 2008 in FIG. 8 is obtained. Thereafter, the superconducting wire structure 2008 shown in FIG. 8 is obtained by the above-described pressurizing method and heat treatment.

The critical current, the mechanical strength, and the porosity of the joint layer 221 of the obtained superconducting wire structure 2008 of FIG. 8 can be measured in the same manner as the methods described in <Measurement of Critical Current>, <Method for Measuring Mechanical Strength of Superconducting Wire Structure in FIG. 1>, and <Method for Measuring Porosity of Joint Layer>.

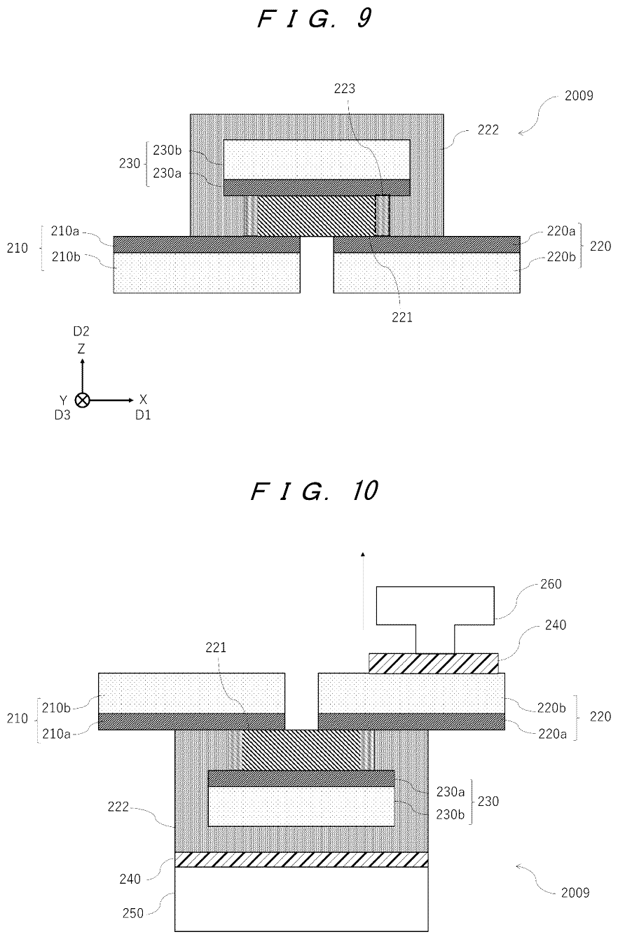

FIG. 9 is a schematic cross-sectional view of a sixth modification of the superconducting wire structure according to the first embodiment. In FIG. 9, the superconducting wire structure 2009 further includes a third wire 230.

The third wire 230 includes a third superconducting layer 230a, and a third substrate 230b. The third superconducting layer 230a is provided on the third substrate 230b. The third superconducting layer 230a and the third substrate 230b respectively may comprise either the same or different materials of the first superconducting layer 210a and the first substrate 210b.

The third superconducting layer 230a is provided to face the first superconducting layer 210a and the second superconducting layer 220a. By providing third wire 230, the energy loss of the superconducting wire structure 2009 can be reduced. This is because when a current flows from the first wire 210 to the second wire 220, the current can pass through the third superconducting layer 230a having a lower resistance.

The connecting member 222 is provided to connect the joint layer 221 to at least one of the first wire 210 and the second wire 220. In FIG. 9, the connecting member 222 is provided to cover the surface of the third wire 230 exposed to the outside of the third wire and the side surface of the joint layer 221; however, for example, the connecting member 222 can be positioned between the first wire 210 and the second wire. In this case, the connecting member 222 is positioned between the first wire 210 and the second wire 220 and is provided to connect the joint layer 221 to at least one of the first wire 210 and the second wire 220.

The connecting member 222 includes a region 223. The region 223 is located in a range of equal or more than 5 um and equal or less than 1 mm from an edge of the joint layer 221 in the first direction from the first wire 210 to the second wire 220.

The superconducting wire structure 2009 in FIG. 9 can be manufactured as follows.

Firstly, the materials for the joint layer 221 are applied on the third superconducting layer 230a with a required thickness and are dried. After the materials are dried, they are baked in oxygen at equal to or more than 700° C. and equal to or less than 850° C., and the joint layer 221 is obtained. Secondly, each superconducting layer of the first wire 210 and the second wire 220 is arranged to face the same direction. Thereafter, the third wire 230 with the joint layer 221 is arranged to face the first superconducting layer 210a and the second superconducting layer 220a through the joint layer 221. Lastly, the connecting member 222 is provided to be in contact with the third substrate 230b.

Thus, a structure for obtaining superconducting wire structure 2009 in FIG. 9 is obtained. Thereafter, the superconducting wire structure 2009 shown in FIG. 9 is obtained by the above-described pressurizing method and heat treatment.

The critical current, the mechanical strength, and the porosity of the joint layer 221 of the obtained superconducting wire structure 2009 of FIG. 9 can be measured in the same manner as the methods described above in <Measurement of Critical Current>, <Method for Measuring Mechanical Strength of Superconducting Wire Structure in FIG. 1>, and <Method for Measuring Porosity of Joint Layer>.

A method for measuring the mechanical strength in FIG. 9 will be described with reference to FIG. 10. Here, FIG. 10 is a schematic cross-sectional view showing a method of measuring the mechanical strength in the example of the superconducting wire structure 2009 shown in FIG. 9.

### <Method for Measuring Mechanical Strength of Superconducting Wire Structure in FIG. 9>

In FIG. 10, firstly, a case in which the third substrate 230b is covered with the connecting member 222 and the position of the third substrate 230b cannot be determined will be described. In this case, as shown in FIG. 10, the third substrate 230b is fixed to the pedestal 250 via the double-sided adhesive tape 240 so that the entire connecting member 222 covering the third substrate 230b is fixed to the pedestal 250. The first substrate 210b or the second substrate 220b is fixed to the tensile device 260 (in the example of FIG. 10 substrate 220b is fixed) in the same manner as the method described above in <Method for Measuring Mechanical Strength of Superconducting Wire Structure in FIG. 6>.

On the other hand, a case in which the third substrate 230b is not fully covered with the connecting member 222 and the position of the third substrate 230b can be determined in FIG. 10 will be described. In this case, the double-sided adhesive tape 240 is prepared in the same manner as <Method for Measuring Mechanical Strength of Superconducting Wire Structure in FIG. 1>, and the third substrate 230b is fixed to the pedestal 250. The first substrate 210b or the second substrate 220b is fixed to the tensile device 260 in the same manner as the method described in <Method for Measuring Mechanical Strength of Superconducting Wire Structure in FIG. 6>.

In the example of FIG. 10, the tensile device 260 may be fixed to either one of the first and second substrate 210b or 220b or both of the first and second substrates 210b and 220b.

After the superconducting wire structure 2009 is fixed to the pedestal 250 and the tensile device 260 by using the double-sided adhesive tape 240, the mechanical strength of the superconducting wire structure 2009 is measured by using the tensile device 260.

The mechanical strength between the first wire 210, the second wire 220, or the third wire 230 and the joint layer 221, can be evaluated by the above-described method.

FIG. 11 is a schematic cross-sectional view of a seventh modification of the superconducting wire structure according to the first embodiment.

The connecting member 222 includes a fifth connecting member 222e and a sixth connecting member 222f. In FIG. 11, the fifth 222e is provided to cover the lower surface of the joint layer 221.

The sixth connecting member 222f is provided to cover the surfaces of the third wire 230 exposed to the outside and the side surfaces of the joint layers 221.

The third wire 230 is preferably covered with the connecting member 222, which can improve the mechanical strength of the superconducting wire structure 2011.

The superconducting wire structure 2011 in FIG. 11 can be manufactured as follows.

After a structure for obtaining the superconducting wire structure 2009 in FIG. 9 as described above is obtained, the connecting member 222e is arranged so that the connecting member 222e is in contact with at least a part of the first wire 210 and at least a part of the second wire 220.

Thus, the structure for obtaining superconducting wire structure 2011 in FIG. 11 is obtained. Thereafter, the superconducting wire structure 2011 in FIG. 11 is obtained by the above-described pressurizing method and heat treatment.

The critical current, the mechanical strength, and the porosity of the joint layer 221 of the obtained superconducting wire structure 2011 of FIG. 11 can be measured in the same manner as the methods described above in <Measurement of Critical Current>, <Method for Measuring Mechanical Strength of Superconducting Wire Structure in FIG. 1>, and <Method for Measuring Porosity of Joint Layer>.

A superconducting wire structure according to a first embodiment includes: a first superconducting wire; a second superconducting wire adjacent to the first superconducting wire; a joint layer electrically connecting the first superconducting wire and the second superconducting wire to each other; and a connecting member connecting the joint layer and at least one of the first superconducting wire and the second superconducting wire to each other. The connecting member has a melting point equal to or more than 900° C. and equal to or less than 1100° C. Thus, the superconducting wire structure having a high critical current and a high mechanical strength can be realized.

### Second Embodiment

A superconducting coil according to a second embodiment includes the superconducting wire structure according to the first embodiment. Hereinafter, the description of a part of the content overlapping the first embodiment may be omitted.

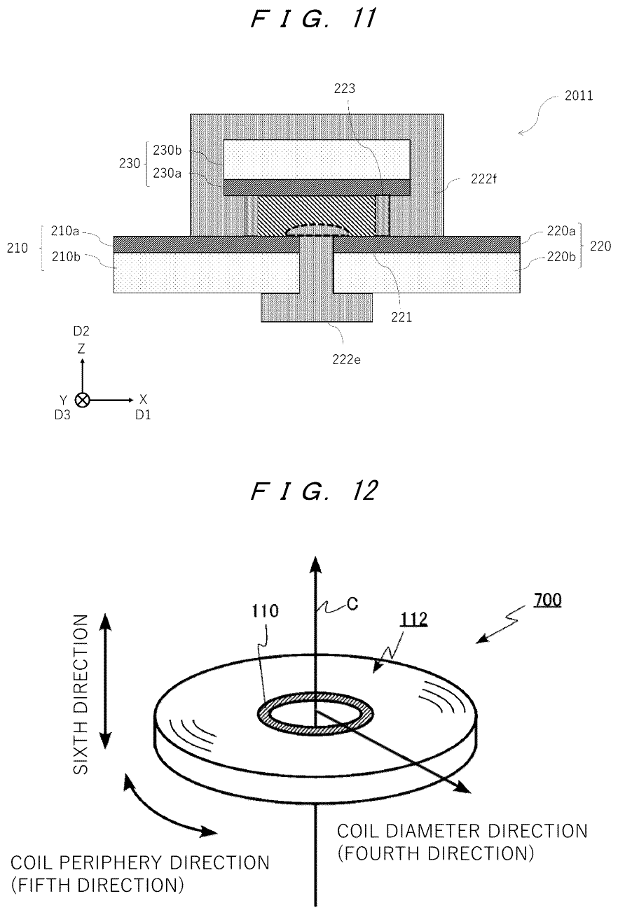

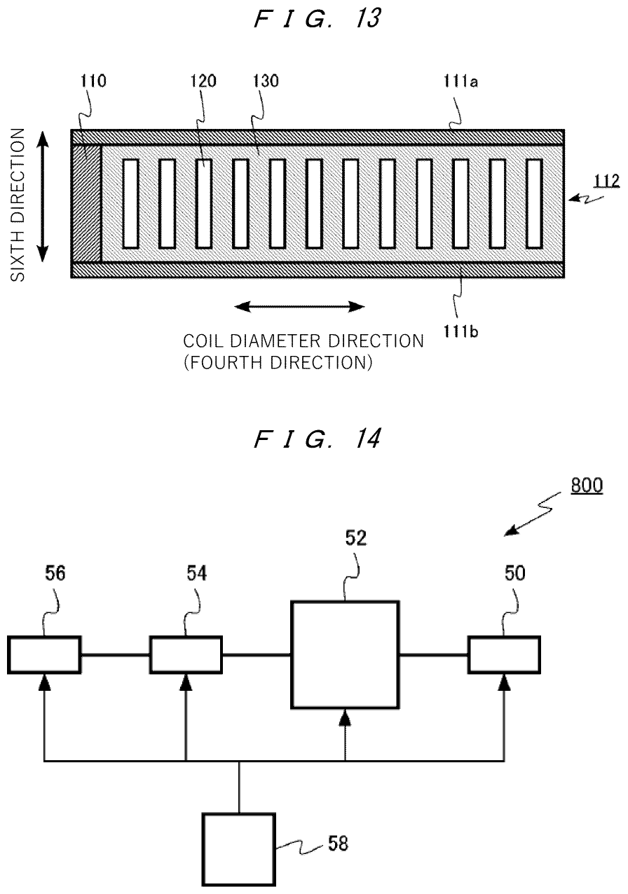

FIG. 12 is a schematic perspective view of the superconducting coil according to the second embodiment. FIG. 13 is a schematic cross-sectional view of the superconducting coil according to the second embodiment.

A superconducting coil 700 according to the second embodiment is used, for example, as a magnetic field generating coil for a superconducting device, such as an NMR, an MRI, a heavy particle beam radiotherapy device, or a superconducting magnetic levitation railway vehicle.

The superconducting coil 700 includes a winding frame 110, a first insulating plate 111a, a second insulating plate 111b, and a winding portion 112. The winding portion 112 has a superconducting wire 120 and an inter-wire layer 130.

FIG. 12 shows a state in which the first insulating plate 111a and the second insulating plate 111b are removed.

The winding frame 110 is formed of fiber-reinforced plastic, for example. The superconducting wire 120 has, for example, a tape shape. As shown in FIG. 12, the superconducting wire 120 is wound around the winding frame 110 in a concentric so-called pancake shape with the winding central axis C as its axis.

The inter-wire layer 130 has a function of fixing the superconducting wire 120. The inter-wire layer 130 has a function of suppressing destruction of the superconducting wire 120 due to vibration during use of the superconducting device or friction therebetween.

The first insulating plate 111a and the second insulating plate 111b are formed of fiber-reinforced plastic, for example. The first insulating plate 111a and the second insulating plate 111b have a function of insulating the winding portion 112 from the outside. The winding portion 112 is disposed between the first insulating plate 111a and the second insulating plate 111b.

The superconducting wire structure 200 according to the first embodiment is used as the superconducting wire 120.

As described above, according to the second embodiment, a superconducting coil having improved characteristics can be realized by providing a superconducting wire having a low electrical resistance and a high mechanical strength.

### Third Embodiment

A superconducting device according to a third embodiment is a superconducting device including the superconducting coil according to the second embodiment. Hereinafter, the description of a part of the content overlapping the first and the second embodiment will be omitted.

FIG. 14 is a block diagram of the superconducting device according to the third embodiment. The superconducting device according to the third embodiment is a heavy particle beam radiotherapy device 800. The heavy particle beam radiotherapy device 800 is an example of the superconducting device.

The heavy particle beam radiotherapy device 800 includes a beam delivery system 50, a synchrotron accelerator 52, a beam transport system 54, an emission system 56, and a control system 58.

The beam delivery system 50 has a function of, for example, generating carbon ions used for treatment and pre-accelerating the carbon ions to be incident on the synchrotron accelerator 52. The beam delivery system 50 includes, for example, an ion source and a linear accelerator.

The synchrotron accelerator 52 has a function of accelerating the carbon ion beam from the beam delivery system 50 to an energy suitable for treatment. The superconducting coil 700 according to the second embodiment is used as the synchrotron accelerator 52.

The beam transport system 54 has a function of transporting the carbon ion beam from the synchrotron accelerator 52 to the emission system 56. The beam transport system 54 includes, for example, a deflection electromagnet.

The emission system 56 has a function of emitting the carbon ion beam from the beam transport system 54 to a patient that is an emission target. The emission system 56 includes, for example, a rotating gantry that allows the carbon ion beam to be emitted from any direction. The superconducting coil 700 according to the second embodiment is used as the rotating gantry.

The control system 58 controls the beam delivery system 50, the synchrotron accelerator 52, the beam transport system 54, and the emission system 56. The control system 58 is, for example, a computer.

In the heavy particle beam radiotherapy device 800 according to the third embodiment, the superconducting coil 700 according to the third embodiment is used as the synchrotron accelerator 52 and the rotating gantry. Therefore, the heavy particle beam radiotherapy device 800 with excellent characteristics is realized.

In the third embodiment, as an example of a superconducting device, the case of the heavy particle beam radiotherapy device 800 has been described. However, the superconducting device may be a nuclear magnetic resonance (NMR) apparatus, a magnetic resonance imaging (MRI) apparatus, or a superconducting magnetic levitation railway vehicle.

### EXAMPLES

### Example 1

Three wires on which a GdBa₂Cu₂O_{δ} layer (oxide superconducting layer) was formed were prepared. The width and length of the two wires were respectively 4 mm and 50 mm, and the width and length of the other wire were respectively 12 mm and 10 mm. 15 mm from the tip of the protective layers for the two long thin wires, and the entire surface of the protective layer for the short thick wire were wet etched using a mixed solution of nitric acid, ammonia, and hydrogen peroxide to expose the oxide superconducting layers (corresponding to the superconducting layers of the first and second wires and the superconducting layer of the third wire)

Next, powders of Gd₂O₃, BaCO₃, and CuO were prepared, appropriately weighed, and then sufficiently mixed. The mixed powder was subjected to a heat treatment at 900° C. to obtain a calcined body. The calcined body was pulverized, and the obtained powder was compression-molded to produce a green compact. The obtained green compact was sintered at 960° C., by which an oxide superconductor having a composition of GdBa₂Cu₃O_{δ} (6≤δ≤7) was manufactured. The obtained oxide superconductor was wet pulverized and then a heat treatment in oxidizing atmosphere was performed at 470° C. to obtain a powder of monocrystalline or polycrystalline particles of GdBa₂Cu₃O_{δ} (6≤δ≤7) having a mean particle size of 5.9 μm.

The obtained powder and a solution mainly including an organic compound containing the same metal element as that of the obtained powder were mixed at a weight ratio of 2:1, and the mixture was applied on the superconducting layer of the third wire. After drying the mixture at 150° C., the product was sintered in oxygen at 800° C.

Firstly, a 10 mm square Ag sheet having a thickness of 50 μm was placed on a pressurizing jig, and the first and the second wires were placed on the Ag sheet with the superconducting layers of each wire facing upward. Secondly, the third wire was placed on the superconducting layers of the first and the second wires with the joint layer facing downward, and another 10 mm square Ag sheet having a thickness of 50 μm was further placed on the third wire. Lastly, another pressurizing jig was placed on the Ag sheet in contact with the third wire. The superimposed wires and Ag sheets were interposed between pressurizing jigs from above and below and pressed with a torque of 4 N·m. A heat treatment was performed at 820° C. in argon containing 500 ppm oxygen, and subsequently another heat treatment was performed at 450° C. in oxygen while the wires and the Ag sheets were interposed between pressurizing jigs.

The sample taken out from the furnace was taken out from the pressurizing jig, and after terminals were attached, the sample was immersed in liquid nitrogen, and the critical current was measured. Another sample prepared in the same manner was used for the mechanical strength and then for the evaluation of the porosity.

### <Method for Measuring Critical Current>

The critical current in the manufactured superconducting wire structure measured by a four terminal measurement method. The terminal was attached to the superconducting wire structure after the connection, and measurement was performed in liquid nitrogen. Since the voltage increases as the resistance increases, the current value at which the voltage starts to increase was defined as the critical current. The obtained values of critical current are shown in Tables 1 and 2 below. The values of critical current shown in Tables 1 and 2 are values indicating how many times they are as much as Comparative Example 1 when the value of Comparative Example 1 is defined as 1.

### <Method for Measuring Mechanical Strength of Superconducting Wire Structure>

The mechanical strength of the superconducting wire structure was evaluated according to Japanese Industrial Standards JIS K 6849 (1994) “Testing methods for tensile strength of adhesive bonds (method of measuring by tensile load perpendicular to the bonding surface)”. In the measurement, firstly, the third substrate was fixed to the pedestal via double-sided adhesive tape so that the entire member covering the third substrate was fixed to the pedestal.

Secondly, the first substrate was fixed to the tensile device via double-sided adhesive tape. The double-sided adhesive tape was prepared so that the width of the tape was 4 mm, which is the same length as the first wire. Further, the length of the tape was a length that enabled strength that did not cause peeling between the pedestal and the first substrate during measurement of the mechanical strength. A carbon tape for SEM observation was used for the double-sided adhesive tape.

Thus, as the operation of the tensile device, the tensile device was operated in a direction away from the first wire fixed to the tensile device via the double-sided adhesive tape. At this time, the operation speed of the tensile device was set to be constant, and the speed was set to be 700 μm/s. The distance by which the tensile device moved until the first wire was completely peeled off from the joint layer was measured as the mechanical strength. The obtained mechanical strengths are shown in Tables 1 and 2. The values of mechanical strength shown in Tables 1 and 2 are values indicating how many times they are as much as Comparative Example 1 when the value of Comparative Example 1 is defined as 1.

### <Method for Measuring Porosity of Joint Layer>

First, the connection portion including the joint layer was embedded in an epoxy-based resin, and the vicinity of the center of the connection portion which was firmly connected was cut by a diamond saw because peeling was concerned at the end of the connection. At this time, the cross section for measuring the porosity of the joint layer was located at a position avoiding the connecting member existing between the joint layer and the first wire or the second wire.

Next, the cut surface was polished with waterproof paper and buffed, and then coated with a conductive material, and the observation positions were determined by SEM. Ion milling was performed on the determined observation positions, and SEM observation was performed after conductive coating was performed.

SEM images of three positions in the cross section were taken at a magnification of about 2500 times. At this time, the joint layer was processed to have three cross sections at positions where the sides corresponding to the long sides of the substantially quadrangular shape of the joint layer in plan view were equally divided into four. The SEM image was binarized to separate particles and voids using ImageJ, and then the areas of the particles and the voids were calculated. The porosity in each cross section was calculated from the ratio of the areas. The porosity obtained in each cross section was averaged to obtain the porosity of the joint layer to be obtained. The obtained porosities of the joint layer are shown in Tables 1 and 2.

### <Method for Measuring Length Along First Direction of Member Entering Joint Layer>

The distance was measured by measuring the connecting member remaining on the first superconducting layer of the first wire after the separation of the joint layer and the first wire.

First, after <Method for Measuring Mechanical Strength of Superconducting Wire Structure> was performed, the joint layer and the first wire were separated from each other.

Thereafter, a mapping image of elements derived from the components of the connecting member was captured by EDX on the surfaces of the first superconducting layer of the first wire. This makes it possible to measure the depth of the connecting member entering the joint layer in the first direction.

When capturing EDX images, first, the area on the surface of the first superconducting layer, where the joint layer existed before separation from the first wire, was divided into five equal parts along the third direction, which intersects with the first direction and is parallel to the surface. The regions at both ends of the five equal parts along the third direction were excluded from the EDX imaging area. The EDX imaging magnification was set to a range where the connecting member with the longest distance along the first direction was within the field of view in each imaging area.

In each EDX image obtained in this way, the average length of the connecting member in the third direction on the surface of the first superconducting layer was determined. The average length of the invasion mentioned above was determined at positions dividing each width of the EDX images along the third direction into six equal parts. By averaging these five lengths obtained from each of the three EDX images, a total of 15 lengths, the length of the connecting member invading the joint layer along the first direction was determined. The obtained lengths along the first direction of the connecting member entering the joint layer are shown in Tables 1 and 2.

### Example 2

A sample was manufactured and evaluated in the same manner as in Example 1, except that a powder of monocrystalline or polycrystalline particles of GdBa₂Cu₃O_{δ} (6≤δ≤7) having a mean particle size of 2.2 μm was used, and the obtained powder and a solution mainly including an organic compound containing the same metal element as that of the obtained powder were mixed at a weight ratio of 1:1.

### Example 3

A sample was manufactured and evaluated in the same manner as in Example 1, except that the thickness of the Ag sheet was 20 μm.

### Example 4

A sample was manufactured and evaluated in the same manner as in Example 1, except that the Ag sheet was only placed under the first wire and the second wire.

### Example 5

The obtained powder described in Example 1 and a solution mainly including an organic compound containing the same metal element as that of the obtained powder were mixed at a weight ratio of 2:1, and the mixture was applied on the Ag sheet instead of the third wire. After drying the mixture at 150° C., the product was sintered in oxygen at 800° C. A sample was manufactured and evaluated in the same manner as in Example 1, except that the obtained joint layer on the Ag sheet was placed on the first and the second superconducting layers and the other Ag sheet was not used.

### Example 6

The first and the second wires with the protective layers separated described in Example 1 were only used as wires, the mixture described in Example 1 was applied on either of the wires. After drying the mixture at 150° C., the product was sintered in oxygen at 800° C. A sample was manufactured and evaluated in the same manner as in Example 1, except that the first wire and the second wire were arranged to face each other.

### Example 7

A sample was manufactured and evaluated in the same manner as in Example 1, except that a Cu sheet was used instead of an Ag sheet.

### Example 8

A sample was manufactured and evaluated in the same manner as in Example 1, except that an Au sheet was used instead of an Ag sheet.

### Example 9

A sample was manufactured and evaluated in the same manner as in Example 1, except that a Ge sheet was used instead of an Ag sheet.

### Example 10

A sample was manufactured and evaluated in the same manner as in Example 1, except that a powder of monocrystalline or polycrystalline particles of GdBa₂Cu₃O_{δ} (6≤δ≤7) having a mean particle size of 1.7 μm was used, and the obtained powder and a solution mainly including an organic compound containing the same metal element as that of the obtained powder were mixed at a weight ratio of 1:1.

### Example 11

A sample was manufactured and evaluated in the same manner as in Example 1, except that a powder of monocrystalline or polycrystalline particles of GdBa₂Cu₃O_{δ} (6≤δ≤7) having a mean particle size of 8.6 μm was used, and the obtained powder and a solution mainly including an organic compound containing the same metal element as that of the obtained powder were mixed at a weight ratio of 1:2.

### Example 12

A sample was manufactured and evaluated in the same manner as in Example 1, except that a powder of monocrystalline or polycrystalline particles of GdBa₂Cu₃O_{δ} (6≤δ≤7) having a mean particle size of 1.3 μm was used, and the obtained powder and a solution mainly including an organic compound containing the same metal element as that of the obtained powder were mixed at a weight ratio of 1:1.

### Example 13

A sample was manufactured and evaluated in the same manner as in Example 1, except that a powder of monocrystalline or polycrystalline particles of GdBa₂Cu₃O_{δ} (6≤δ≤7) having a mean particle size of 11.0 μm was used, and the obtained powder and a solution mainly including an organic compound containing the same metal element as that of the obtained powder were mixed at a weight ratio of 1:2.

### Comparative Example 1

A sample was manufactured and evaluated in the same manner as in Example 1, except that the Ag sheet was not placed.

### Comparative Example 2

A sample was manufactured and evaluated in the same manner as in Example 2, except that the Ag sheet was not placed.

### Comparative Example 3

A sample was manufactured and evaluated in the same manner as in Example 1, except that the Ag sheet was not placed and the entire connection portion was sealed with solder at 300° C. after the sample was sintered.

### Comparative Example 4

A sample was manufactured and evaluated in the same manner as in Example 1, except that the Ag sheet was not placed and the entire connection portion was fixed by pressure bonding with Ag sheet at room temperature after the sample was sintered.

### Comparative Example 5

A sample was manufactured and evaluated in the same manner as in Example 1, except that an Al sheet was used instead of the Ag sheet.

### Comparative Example 6

A sample was manufactured and evaluated in the same manner as in Example 1, except that a Pt sheet was used instead of the Ag sheet.

The values of critical current, the mechanical strength, the porosity of the joint layer, and the length along the first direction of the connecting member entering the joint layer in all examples and comparative examples are shown in Tables 1 and 2.

TABLE 1

| | Melting | | Porosity | Length of | | |
|---|---|---|---|---|---|---|
| | point of | | of joint | member entering | Critical | Mechanical |
| | member [° C.] | Member | layer [%] | joint layer [μm] | current | strength |
| Example 1 | 961.8 | Ag | 44 | 325 | 1.2 | 3.5 |
| Example 2 | 961.8 | Ag | 36 | 277 | 1.9 | 3.6 |
| Example 3 | 961.8 | Ag | 44 | 218 | 1.2 | 4.2 |
| Example 4 | 961.8 | Ag | 44 | 230 | 1.2 | 3.3 |
| Example 5 | 961.8 | Ag | 44 | 230 | 1.1 | 3.5 |
| Example 6 | 961.8 | Ag | 44 | 325 | 1.2 | 3.5 |
| Example 7 | 1085 | Cu | 44 | 38 | 1.2 | 3.3 |
| Example 8 | 1064 | Au | 44 | 121 | 1.2 | 3.4 |
| Example 9 | 938.2 | Ge | 44 | 782 | 1.2 | 3.7 |
| Example 10 | 961.8 | Ag | 15 | 97 | 1.6 | 3.1 |
| Example 11 | 961.8 | Ag | 65 | 421 | 1 | 4 |
| Example 12 | 961.8 | Ag | 5 | 76 | 1.3 | 3 |
| Example 13 | 961.8 | Ag | 80 | 596 | 1 | 4.1 |

TABLE 2

| | Melting | | Porosity | Length of | | |
|---|---|---|---|---|---|---|
| | point of | | of joint | member entering | Critical | Mechanical |
| | member [° C.] | Member | layer [%] | joint layer [μm] | current | strength |
| Comparative | — | — | 44 | — | 1 | 1 |
| Example 1 | | | | | | |
| Comparative | — | — | 36 | — | 1.2 | 1.1 |
| Example 2 | | | | | | |
| Comparative | — | — | 44 | — | 0.6 | 3.2 |
| Example 3 | | | | | | |
| Comparative | — | — | 50 | — | 0.1 | 3.5 |
| Example 4 | | | | | | |
| Comparative | 660 | Al | 44 | 1500 | 0.5 | 3.6 |
| Example 5 | | | | | | |
| Comparative | 1769 | Pt | 44 | 0 | 1 | 1 |
| Example 6 | | | | | | |

In contrast to Comparative Examples 1 to 4 in which the connection started to peel off when the sample was taken out from the pressurizing jigs, all Examples 1 to 6 in which the Ag sheet was provided have improved characteristics of the critical current and the mechanical strength. In particular, in Example 2 in which a powder having a small mean particle size was used, the porosity of the joint layer was low, but the contact area between the particles or between the particle and the superconducting layer of the wire increased. Thus, the critical current was high in Example 2.

In Example 3 using the thin Ag sheet, the Ag easily entered the joint layer, and the mechanical strength was further improved.

On the other hand, in Comparative Example 3 in which the connection portion was sealed with solder, the powder in the joint layer and the superconducting layer in the wire were deteriorated by heat, and the mechanical strength was high, but the critical current was low. Similarly, in Comparative Example 4 in which the connection portion was fixed by pressure bonding with the Ag sheet at room temperature, the structure in the joint layer collapsed by the pressure, and the connection between the particles in the joint layer and the superconducting layer of the wire peeled off, and the mechanical strength was high, but the critical current was low.

It was found that the characteristics of the critical current and the mechanical strength were high also in Examples 7 to 9 in which Cu, Au, and Ge having a melting point of 900° C. to 1100° C. were used instead of the Ag sheet.

On the other hand, in Comparative Example 5 using the Al sheet having a melting point of less than 900° C., Al reacted with the GdBCO material in the joint layer, and the mechanical strength was high, but the critical current was low. In Comparative Example 6 using the Pt sheet having a melting point of more than 1100° C., it was found that the mechanical strength was low because the Pt sheet was not softened.

In the Examples 10 and 11 in which the porosities were 10% to 70%, the characteristics of the critical current and the mechanical strength were high, and the characteristics in Example 12 where the porosity was less than 10% and in Example 13 where the porosity was more than 70% were high as well.

In addition, in Comparative Example 5 using an Al sheet having a melting point of 660° C. instead of the Ag sheet, it was found that the melting of the connecting member excessively progressed, and the length of the connecting member entering the joint layer was increased compared to Example 1. Thus, the connecting member pushed the particles composing the joint layer and this made it unable to obtain a sufficient connection area and to provide a sufficient current path. Hence, it was found that the critical current in Comparative Example 5 was lower than Example 1.

In addition, in Comparative Example 6 in which the Pt sheet having a melting point of 1769° C. was used instead of the Ag sheet, the connecting member was not sufficiently melted, and the connecting member did not enter the joint layer. It was found that the mechanical strength as the superconducting wire structure in Comparative Example 6 was lower than Example 1.

While certain embodiments have been described, these embodiments have been presented by way of example only, and are not intended to limit the scope of the inventions. Indeed, the superconducting layer joint structure, the superconducting wire structure, the superconducting coil, and the superconducting device described herein may be embodied in a variety of other forms; furthermore, various omissions, substitutions and changes in the form of the devices and methods described herein may be made without departing from the spirit of the inventions. The accompanying claims and their equivalents are intended to cover such forms or modifications as would fall within the scope and spirit of the inventions.

Hereinafter, some aspects according to the embodiments will be described.

[1] A superconducting wire structure, comprising:
a first superconducting wire;
a second superconducting wire adjacent to the first superconducting wire;
a joint layer electrically connecting the first superconducting wire and the second superconducting wire to; and
a connecting member connecting the joint layer and at least one of the first superconducting wire and the second superconducting wire,
wherein the connecting member has a melting point equal to or more than 900° C. and equal to or less than 1100° C.

[2] The superconducting wire structure according to [1],
wherein a porosity of the joint layer is equal to or more than 10% and equal to or less than 70%.

[3] The superconducting wire structure according to [1] or [2],
wherein the connecting member includes at least one selected from: a first region existing between the joint layer and the first superconducting wire; and a second region existing between the joint layer and the second superconducting wire,
the first region and the second region satisfy at least one of a first condition and a second condition,
in the first condition, in a first direction crossing a direction from the first superconducting wire to the joint layer and along the first superconducting wire, the first region is located in a range of equal or more than 5 um and equal or less than 1 mm from an end of the first superconducting wire,
in the second condition, in the first direction, the second region is located in a range of equal or more than 5 um and equal or less than 1 mm from an end of the second superconducting wire.

[4] The superconducting wire structure according to [1] or [2],
wherein in a first direction crossing a direction from the first superconducting wire to the joint layer and along the first superconducting wire, the connecting member is located in a range of equal or more than 5 um and equal or less than 1 mm from an edge of the joint layer.

[5] The superconducting wire structure according to any one of [1] to [4],
wherein the connecting member includes at least one selected from the group including: Ag, Cu, Au, and Ge.

[6] The superconducting wire structure according to any one of [1] to [5],
further comprising a third superconducting wire,
wherein the joint layer is located between the first superconducting wire and the third superconducting wire, and between the second superconducting wire and the third superconducting wire,
the joint layer electrically connects the first superconducting wire and the third superconducting wire, and the second superconducting wire and the third superconducting wire.

[7] The superconducting wire structure according to [6],
wherein a porosity of the joint layer is equal to or more than 10% and equal to or less than 70%.

[8] The superconducting wire structure according to claim [6] or [7],
wherein the connecting member includes at least one selected from: a first region existing between the joint layer and the first superconducting wire; and a second region existing between the joint layer and the second superconducting wire,
the first region and the second region satisfy at least one of a third condition and a fourth condition,
in the third condition, in a first direction crossing a direction from the first superconducting wire to the joint layer and along the first superconducting wire, the first region is located in a range of equal or more than 5 um and equal or less than 1 mm from an edge of the first superconducting wire,
in the fourth condition, in the first direction, the second region is located in a range of equal or more than 5 um and equal or less than 1 mm from an edge of the second superconducting wire.

[9] The superconducting wire structure according to any one of [6] to [8],
wherein in a first direction crossing a direction from the first superconducting wire to the joint layer and along the first superconducting wire, the connecting member is located in a range of equal or more than 5 um and equal or less than 1 mm from an edge of the joint layer.

[10] The superconducting wire structure according to any one of [6] to [9],
wherein the connecting member includes at least one selected from the group including: Ag, Cu, Au, and Ge.

[11] A superconducting coil, comprising:
the superconducting wire structure according to any one of [1] to [10].

[12] A superconducting device, comprising:
the superconducting coil according to [11].

What is claimed is:

1. A superconducting wire structure comprising:
a first superconducting wire;
a second superconducting wire adjacent to the first superconducting wire;
a joint layer electrically connecting the first superconducting wire and the second superconducting wire; and
a connecting member connecting the joint layer and at least one of the first superconducting wire and the second superconducting wire,
wherein the connecting member has a melting point equal to or more than 900° C. and equal to or less than 1100° C.

2. The superconducting wire structure according to claim 1,
wherein a porosity of the joint layer is equal to or more than 10% and equal to or less than 70%.

3. The superconducting wire structure according to claim 1,
wherein the connecting member includes at least one selected from: a first region existing between the joint layer and the first superconducting wire; and a second region existing between the joint layer and the second superconducting wire,
the first region and the second region satisfy at least one of a first condition and a second condition,
in the first condition, in a first direction crossing a direction from the first superconducting wire to the joint layer and along the first superconducting wire, the first region is located in a range of equal or more than 5 um and equal or less than 1 mm from an end of the first superconducting wire,
in the second condition, in the first direction, the second region is located in a range of equal or more than 5 um and equal or less than 1 mm from an end of the second wire.

4. The superconducting wire structure according to claim 1,
wherein in a first direction crossing a direction from the first superconducting wire to the joint layer and along the first superconducting wire, the connecting member is located in a range of equal or more than 5 um and equal or less than 1 mm from an edge of the joint layer.

5. The superconducting wire structure according to claim 1,
wherein the connecting member includes at least one selected from the group including: Ag, Cu, Au, and Ge.

6. The superconducting wire structure according to claim 1,
further comprising a third superconducting wire,
wherein the joint layer is located between the first superconducting wire and the third superconducting wire, and between the second superconducting wire and the third superconducting wire,
the joint layer electrically connects the first superconducting wire and the third superconducting wire, and the second superconducting wire and the third superconducting wire.

7. The superconducting wire structure according to claim 6,
wherein a porosity of the joint layer is equal to or more than 10% and equal to or less than 70%.

8. The superconducting wire structure according to claim 6,
wherein the connecting member includes at least one selected from: a first region existing between the joint layer and the first superconducting wire; and a second region existing between the joint layer and the second superconducting wire,
the first region and the second region satisfy at least one of a third condition and a fourth condition,
in the third condition, in a first direction crossing a direction from the first superconducting wire to the joint layer and along the first superconducting wire, the first region is located in a range of equal or more than 5 um and equal or less than 1 mm from an edge of the first wire,
in the fourth condition, in the first direction, the second region is located in a range of equal or more than 5 um and equal or less than 1 mm from an edge of the second wire.

9. The superconducting wire structure according to claim 6,
wherein in a first direction crossing a direction from the first superconducting wire to the joint layer and along the first superconducting wire, the connecting member is located in a range of equal or more than 5 um and equal or less than 1 mm from an edge of the joint layer.

10. The superconducting wire structure according to claim 6,
wherein the connecting member includes at least one selected from the group including: Ag, Cu, Au, and Ge.

11. A superconducting coil, comprising:
the superconducting wire structure according to claim 1.

12. A superconducting device, comprising:
the superconducting coil according to claim 11.
